(19) United States
(12) Patent Application Publication
(10) Pub. No.: US 2021/0068668 A1
(43) Pub. Date: Mar. 11, 2021

(54) ELECTRONIC DEVICE AND METHOD FOR OBTAINING VITAL SIGN

(71) Applicant: Samsung Electronics Co., Ltd., Suwon-sis (KR)

(72) Inventors: Kostyantyn SLYUSARENKO, Kyiv (UA); Illya DEGTYARENKO, Kyiv (UA); Andrii OMELCHENKO, Kyiv (UA)

(21) Appl. No.: 16/925,685

(22) Filed: Jul. 10, 2020

(30) Foreign Application Priority Data
Sep. 6, 2019 (KR) 10-2019-0110647

(51) Int. Cl. A61B 5/0205; A61B 5/00

(52) U.S. Cl. CPC A61B 5/0205; A61B 5/021; A61B 5/7278; A61B 5/742

(57) ABSTRACT

An electronic device and a method for obtaining a vital sign using the same are provided. The electronic device includes a memory to store instructions, and at least one processor connected to the memory. The instructions, which when executed, configure the at least one processor to receive a sensing signal related to a user's breathing from a sensor, obtain a first pattern related to a change in the respiration of the user over time, and a second pattern related to a change in saturation of peripheral oxygen of the user over time based on the sensing signal, and determine an oxygen delivery time (ODT), based on a comparison between the first pattern and the second pattern.

### CROSS-REFERENCE TO RELATED APPLICATION(S)

This application is based on and claims priority under 35 U.S.C. § 119 of a Korean patent application number 10-2019-0110647, filed on Sep. 6, 2019, in the Korean Intellectual Property Office, the disclosure of which is incorporated by reference herein in its entirety.

### BACKGROUND

### 1. Field

The disclosure relates to an electronic device and a method for obtaining a vital sign using the same.

### 2. Description of Related Art

Recently, the trend of using healthcare services (monitoring, diagnosis, management, etc.) through popular electronic devices (e.g., smartphones, wearable devices) has been spreading.

However, data related to healthcare services measurable by electronic devices is limited, accuracy of measurement is not high, and the accuracy of and user satisfaction with healthcare services using measurement data may still be insufficient. Accordingly, attempts have been made to improve the accuracy of healthcare services and user satisfaction therewith using new vital-sign parameters.

The above information is presented as background information only to assist with an understanding of the disclosure. No determination has been made, and no assertion is made, as to whether any of the above might be applicable as prior art with regard to the disclosure.

### SUMMARY

As one of vital-sign parameters for healthcare services, there may be a viewpoint considering oxygen delivery time (ODT). The oxygen delivery time is directly related to blood flow velocity, is a key parameter required for estimation of various cardiovascular system parameters (e.g., blood pressure, blood viscosity, etc.), and may be used to monitor, diagnose, or manage a user's state of health, including detection of various cardiovascular diseases.

Solutions to measure vital signs parameters such as oxygen delivery time can only be provided using specialized medical device equipment (e.g., Doppler ultrasound equipment, magnetic resonance imaging (MRI) equipment), and may be difficult to implement on popular electronic devices.

In addition, even if a solution for measuring the oxygen delivery time is implemented in an electronic device, the accuracy of measurement data may be low.

Aspects of the disclosure are to address at least the above-mentioned problems and/or disadvantages and to provide at least the advantages described below. Accordingly, an aspect of the disclosure is to provide an electronic device and a method for obtaining a vital sign using the same, which enables the user to acquire vital-sign parameters for a health care service in a simple and user-friendly manner using an electronic device without specialized medical devices.

Another aspect of the disclosure is to provide an electronic device and a method for obtaining a vital sign using the same, which realize increased precision in obtaining a vital-sign parameter using the electronic device.

Additional aspects will be set forth in part in the description which follows and, in part, will be apparent from the description, or may be learned by practice of the presented embodiments.

Various embodiments may provide an electronic device and a method for obtaining a vital sign using the same, which improves the accuracy of, and user satisfaction with, a healthcare service using a vital-sign parameter obtained using the electronic device.

In accordance with an aspect of the disclosure, an electronic device is provided. The electronic device includes a memory to store instructions, and at least one processor connected to the memory. The instructions, which when executed, configure the at least one processor to receive a sensing signal related to a user's respiration from a sensor, obtain a first pattern related to a change in respiration of the user over time, and a second pattern related to a change in saturation of peripheral oxygen of the user over time, based on the sensing signal, and determine an oxygen delivery time (ODT), based on a comparison between the first pattern and the second pattern.

In accordance with another aspect of the disclosure, a method for obtaining a vital sign by an electronic device is provided. The method includes an operation of receiving a sensing signal related to a user's respiration from a sensor connected to the electronic device, an operation of obtaining a first pattern related to a change in respiration of the user over time, and a second pattern related to a change in saturation of peripheral oxygen of the user over time, based on the sensing signal, and an operation of determining an oxygen delivery time (ODT), based on a comparison between the first pattern and the second pattern.

A storage medium according to various embodiments, may include a non-transitory computer-readable recording medium. The non-transitory computer-readable recording medium may have recorded thereon at least one program comprising commands, which when executed, configure at least one processor of an electronic device to receive a sensing signal related to a user's respiration from a sensor, obtain a first pattern related to a change in respiration of the user over time and, a second pattern related to a change in saturation of peripheral oxygen of the user over time, based on the sensing signal, and determine an oxygen delivery time (ODT), based on a comparison between the first pattern and the second pattern.

According to various embodiments, the vital-sign parameters for healthcare services can be obtained in a simple and user-friendly manner using an electronic device without a specialized medical device.

According to various embodiments, precision can be improved in obtaining a vital-sign parameter using an electronic device.

According to various embodiments, accuracy of, and user satisfaction with, healthcare service can be improved using vital-sign parameters obtained using an electronic device.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a view illustrating a method for obtaining a vital sign according to an embodiment of the disclosure;

FIG. 2A is a view illustrating a method for obtaining a vital sign according to an embodiment of the disclosure;

FIG. 2B is a view illustrating a method for obtaining a vital sign according to an embodiment of the disclosure;

FIG. 2C is a view illustrating a method for obtaining a vital sign according to an embodiment of the disclosure;

FIG. 3 is a block view of an electronic device according to an embodiment of the disclosure;

FIG. 4 is a view illustrating the use of an electronic device according to an embodiment of the disclosure;

FIG. 5A is a graph illustrating an operation of an electronic device according to an embodiment of the disclosure;

FIG. 5B is a graph illustrating an operation of an electronic device according to an embodiment of the disclosure;

FIG. 5C is a graph illustrating an operation of an electronic device according to an embodiment of the disclosure;

FIG. 5D is a graph illustrating an operation of an electronic device according to an embodiment of the disclosure;

FIG. 6A is a flowchart illustrating an operation of obtaining a vital sign according to an embodiment of the disclosure;

FIG. 6B is a flowchart illustrating an operation of obtaining a vital sign according to an embodiment of the disclosure;

FIG. 7A is a graph illustrating a vital-sign acquisition algorithm according to an embodiment of the disclosure;

FIG. 7B is a graph illustrating a vital-sign acquisition algorithm according to an embodiment of the disclosure;

FIG. 8 is a graph illustrating a vital-sign acquisition algorithm according to an embodiment of the disclosure;

FIG. 9 is a view illustrating a method of using a vital-sign parameter obtained according to an embodiment of the disclosure;

FIG. 10 is a view illustrating a method of using a vital-sign parameter obtained according to an embodiment of the disclosure;

FIG. 11A is a view illustrating a method of using a vital-sign parameter obtained according to an embodiment of the disclosure;

FIG. 11B is a view illustrating a method of using a vital-sign parameter obtained according to an embodiment of the disclosure;

FIG. 12 is a view illustrating a method of using a vital-sign parameter obtained according to an embodiment of the disclosure;

FIG. 13 is a view illustrating a method of using a vital-sign parameter obtained according to an embodiment of the disclosure; and

FIG. 14 is a block view illustrating an electronic device in a network environment according to an embodiment of the disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Among the various vital-sign parameters required for healthcare services, there may be a parameter that is difficult to measure simply using a sensor. In order to measure these parameters, professional medical equipment may be required.

This document discloses embodiments for enabling precise measurement of vital-sign parameters required for healthcare services using an electronic device (e.g., smartphones, wearable devices (e.g. smartwatches, smart rings, finger clips), headsets (or ear clips), etc.) without a specialized medical device in a simple and user-friendly manner.

In various embodiments, the vital-sign parameter to be measured may be the oxygen delivery time. The vital-sign parameter to be measured may be a cardiovascular system parameter (e.g., blood pressure, etc.) related to the oxygen delivery time. The cardiovascular system parameters may be measured based on the oxygen delivery time.

In various embodiments, a single sensor may be used to measure the oxygen delivery time. At least one sensor having two or more wavelengths (e.g., a multiple-wavelength photoplethysmogram (PPG) sensor) may be used to measure the oxygen delivery time.

In various embodiments, as basic data for measuring the oxygen delivery time, a plurality of (e.g., two or more) patterns may be obtained from one sensor. The patterns may be obtained from a sensing signal of a sensor, and may be different types of patterns related to parameters required in order to measure the oxygen delivery time. For example, the parameters may include saturation of peripheral oxygen (SpO₂) and respiration. The patterns may include a first pattern representing a respiration pattern and a second pattern representing a saturation of peripheral oxygen pattern. For example, the first pattern and the second pattern may be obtained based on the sensing signal from a sensor. The first pattern may be a time-scale signal of saturation of peripheral oxygen (a signal indicating a change in saturation of peripheral oxygen values over time) generated based on the sensing signal. The second pattern may be a respiration time-scale signal (a signal indicating a change in respiration values over time) generated based on the sensing signal. For example, each pattern may be an output of a sensor, a signal, information, or data obtained (e.g., extracted, generated, signal processed or processed) from the output of the sensor.

In various embodiments, one sensor may be used as a single source to provide a sensing signal required for the measurement of oxygen delivery time. The sensor may be a multiple-wavelength PPG sensor. The electronic device may determine the oxygen delivery time, based on the comparison (e.g., pattern matching) between the first pattern and the second pattern, obtained from the sensor.

In various embodiments, a plurality of (e.g., two or more) patterns (e.g., respiration patterns and saturation of peripheral oxygen patterns) required to determine the oxygen delivery time may be patterns obtained from a single sensor (or a sensing signal thereof), which is a single source. The patterns may be obtained in response to a single user input to the sensor. The patterns may be synchronized patterns. Because the patterns are obtained from a single source, separate synchronization means may be unnecessary. Due to the use of a single source, a plurality of patterns (e.g., respiration patterns and saturation of peripheral oxygen patterns) required for the measurement of the oxygen delivery time are registered (stored) at the same time, or the same timestamp is applied to the plurality of patterns, so that the plurality of patterns can be synchronized.

Various embodiments may enable automatic synchronization of patterns necessary for the measurement of the oxygen delivery time by measuring the oxygen delivery time, based on the output from the same sensor. For this reason, the measurement principle of the oxygen delivery time can be simplified, and the precision of measurement can be increased.

Various embodiments may be capable of obtaining patterns reflecting changes in signal values over time and determining oxygen delivery time, based on comparisons between the patterns (e.g., pattern matching), instead of using a signal value at one time in a fragmentary manner. By doing so, it is possible to accurately identify the user's state of health and to use the same to provide a healthcare service and improve user satisfaction therewith.

Hereinafter, various embodiments will be described with reference to the accompanying drawings.

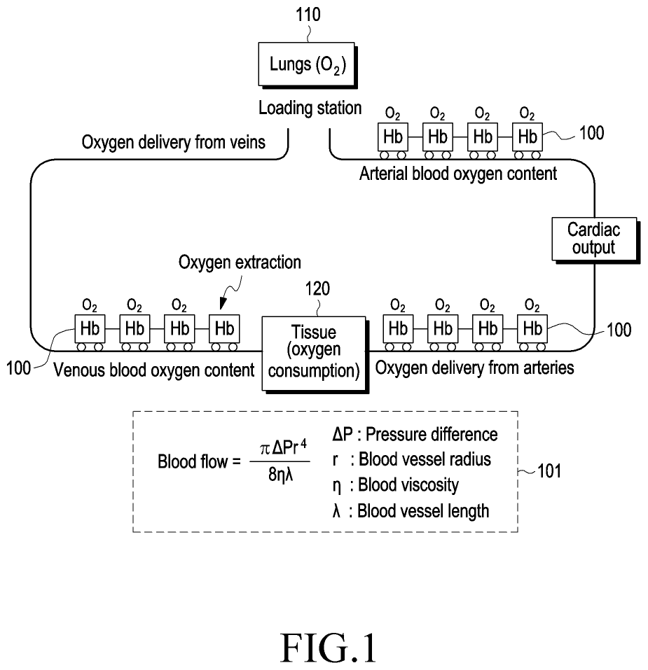

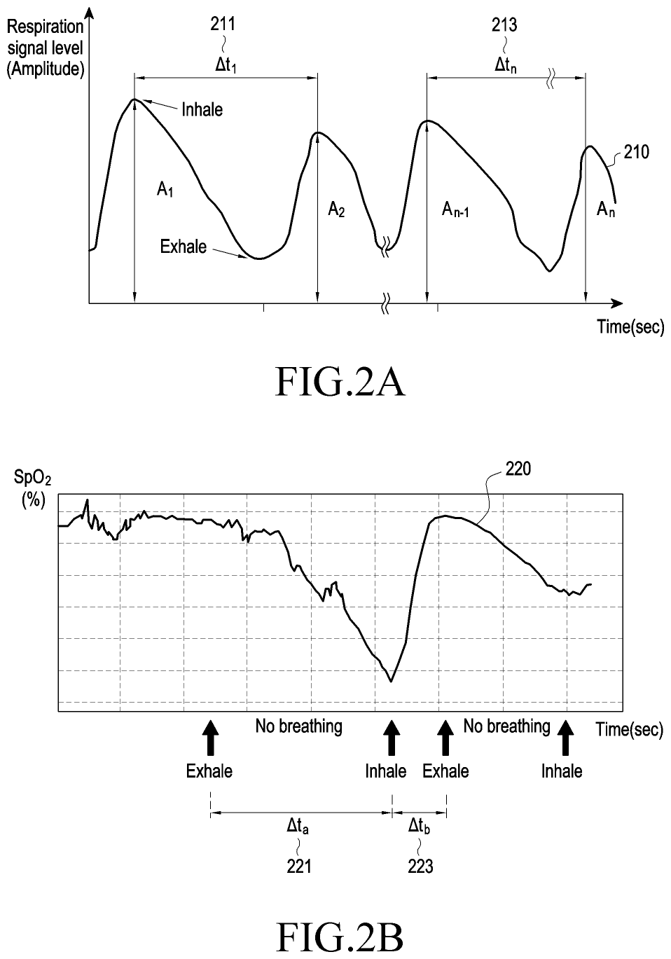

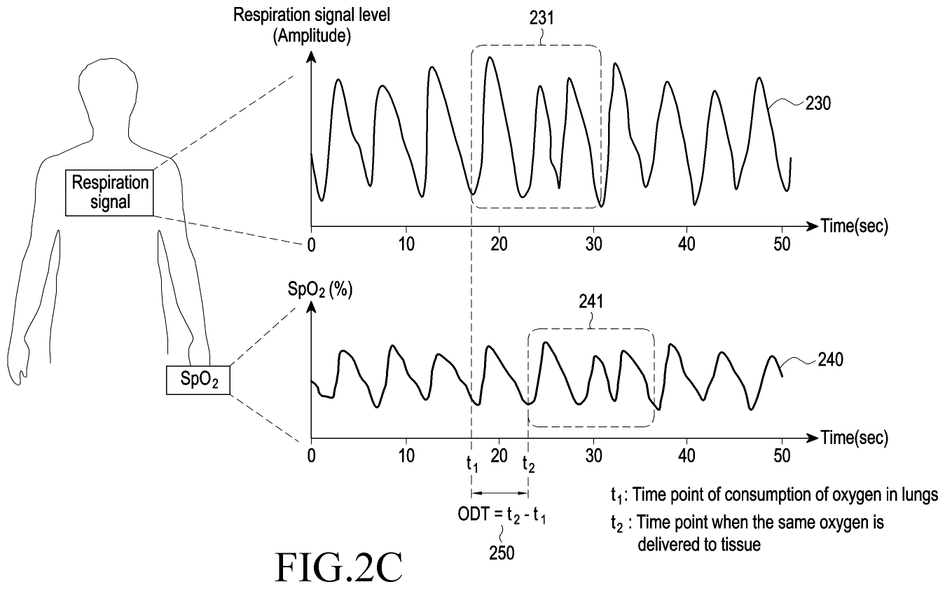

FIG. 1 is a view illustrating a method for obtaining a vital sign according to an embodiment of the disclosure, FIG. 2A is a view illustrating a method for obtaining a vital sign according to an embodiment of the disclosure, FIG. 2B is a view illustrating a method for obtaining a vital sign according to an embodiment of the disclosure, and FIG. 2C is a view illustrating a method for obtaining a vital sign according to an embodiment of the disclosure.

Referring to FIG. 1 is a view for describing the concept of blood flow and oxygen delivery time among vital-sign parameters required for healthcare services.

“Blood flow” may be determined using Equation 101 shown in FIG. 1. According to the Equation of FIG. 1, the blood flow may be determined based on a difference in pressure (blood pressure), a radius of a blood vessel, a viscosity of blood, and a length of a blood vessel.

“Oxygen delivery time” may mean the time taken for oxygen to propagate from the lungs 110 to tissue 120 (e.g., fingers) by blood flow.

The blood flow and oxygen delivery time may be used as key parameters to estimate various cardiovascular system parameters (e.g., blood pressure, blood viscosity, vascular status).

“Oxygen delivery” may be referred to as “train with boxcars” 100. Here, the total oxygen content (or arterial blood oxygen content) may represent oxygen carried on the hemoglobin “boxcars”. The “train engine” may indicate cardiac output. The oxygen delivery time may be an inverse value of a speed of “train 100”.

The global oxygen delivery may mean the amount of oxygen delivered from the lungs 110 to the entire body. This may be determined by the total oxygen content (or arterial blood oxygen content) and cardiac output.

According to various embodiments, a natural oxygen consumption index (or an oxygen consumption detection index, for example, a respiration pattern and a saturation of peripheral oxygen pattern) associated with a user's respiration may be sensed and used to measure an oxygen delivery time (e.g., the same oxygen from the lungs to tissues). According to various embodiments, it is possible to easily and accurately identify the oxygen delivery time, which is used as a vital-sign parameter for a healthcare service, based on a natural oxygen detection index.

According to various embodiments, two or more respiration-related patterns (e.g., patterns related to a user's respiration behavior, such as respiration patterns and saturation of peripheral oxygen patterns) may be identified using a single sensor, without an additional sensor for measuring respiration. According to various embodiments, because a plurality of (e.g., two or more) respiration-related patterns are generated in response to a simple user input to a sensor of an electronic device, or from the output of a single sensor, respiration-related patterns may be obtained in an already synchronized state. For this reason, separate synchronization operations for a plurality of patterns required for measuring the oxygen delivery time may be omitted. In addition, it is possible to simplify the measurement principle of the oxygen delivery time using a plurality of respiration-related patterns and to increase the precision of measurement.

FIGS. 2A and 2B are graphs illustrating respiration patterns 210 (e.g., breathing patterns) and saturation of peripheral oxygen patterns 220 as patterns related to the user's respiration.

As parameters related to breathing of a user, respiration and saturation of peripheral oxygen (SpO₂) may be considered.

The saturation of peripheral oxygen (SpO₂) may indicate peripheral capillary oxygen saturation or the amount of oxygen in the blood. The saturation of peripheral oxygen may correspond to the ratio of oxygenated hemoglobin (oxygen-containing hemoglobin) and blood hemoglobin (oxygenated and non-oxygenated hemoglobin). The saturation of peripheral oxygen may be an estimated value of arterial saturation of peripheral oxygen, indicating the amount of oxygenated hemoglobin in the blood.

In various embodiments, patterns related to saturation of peripheral oxygen (SpO₂) and respiration may be used as an oxygen-sensing indicator for measurement of oxygen delivery time. Both of these patterns are based on the same physiological breathing procedure (the user's breathing) with unique amplitude and phase patterns.

Referring to FIG. 2A, the breathing pattern 210 may be a pattern related to changes in breathing over time. The breathing pattern 210 may be represented by a breathing time-scale signal indicating a change in breathing values over time, as shown in FIG. 2A. The X-axis may be time and the Y-axis may be the magnitude (or strength or intensity) of the breathing signal. The signal amplitude (amplitude) of the Y-axis may be defined by the breathing depth. Sections of the X-axis (e.g., 211, 213) may be defined by breathing periodicity. The breathing pattern 210 shown in FIG. 2A may be extracted from the output of the sensor (e.g., PPG signal).

Referring to FIG. 2B, the saturation of peripheral oxygen pattern 220 may be a pattern related to a change in saturation of peripheral oxygen of a user over time. The saturation of peripheral oxygen pattern 220 may be represented by an oxygen saturation time-scale signal indicating changes in saturation of peripheral oxygen values over time, as shown in FIG. 2B. The signal size (or signal strength or intensity) of the Y-axis may be a rate of change (%) of saturation of peripheral oxygen (SpO₂). The saturation of peripheral oxygen pattern 220 may be generated from the output of the sensor (e.g., a PPG signal). Changes in saturation of peripheral oxygen in the blood during the breathing period between inhalation and exhalation (e.g., 223), or the apnea interval, in which breathing is stopped (e.g., 221), may be examined.

FIG. 2C is a view for illustratively explaining a comparison (e.g., pattern matching) between a respiration pattern and a saturation of peripheral oxygen pattern.

In various embodiments, the oxygen delivery time may be measured based on the time difference between saturation of peripheral oxygen (e.g., an oxygen saturation time-scale signal) 240 and a respiration pattern (e.g., a respiration time-scale signal) 230. The time difference may also be referred to as a time delay or time shift.

The respiration pattern 230 and saturation of peripheral oxygen pattern 240 may be measured at the same oxygen delivery point (e.g., the user's finger). The two patterns 230 and 240 have the same period (or interval) (e.g., 50 seconds) as the response to a single user input to a single sensor (e.g., a multiple-wavelength PPG sensor) (e.g., a user's finger touching the sensor 301).

The oxygen delivery time may be determined based on a comparison between a respiration pattern 230 and a saturation of peripheral oxygen pattern 240 measured at the same oxygen delivery point (e.g., a user's finger). The time difference between the two patterns 230 and 240 may be identified through comparison of the two patterns 230 and 240, and the oxygen delivery time may be determined corresponding to the time difference.

Pattern matching between the two patterns 230 and 240 may be performed so that sections corresponding to each other (e.g., sections having the same (or similar) pattern) may be identified. For example, the first section 231 of the respiration pattern 230 and the second section 241 of the saturation of peripheral oxygen pattern 240 may be identified. The pattern matching the first section 231 and the second section 241 may be generated by the same physiological breathing procedure (the user's breathing) The pattern matching the first section 231 and the second section 241 may be generated as a response (result) to the user's same breathing motion.

The oxygen delivery time (ODT) may be identified based on a time difference between two sections corresponding to each other among the respiration pattern 230 and the saturation of peripheral oxygen pattern 240. In the example of FIG. 2C, the oxygen delivery time 250 may be determined as “second time point t2−first time point t1”. The first time point t1 may be the time point of oxygen consumption in the lungs. The second time point t2 may be the time point at which the same oxygen is delivered to tissue.

Various known algorithms may be used for pattern matching between two patterns. For example, a pattern recognition algorithm for calculating correlation between two signals, a shape-based similarity recognition algorithm such as dynamic time warping, an elasticity-matching algorithm, and an algorithm recognizing a time difference between two peaks in two corresponding signals may be used. The above algorithms are merely examples for explanation, not limitation, and may be applied, or modified in various ways for pattern matching.

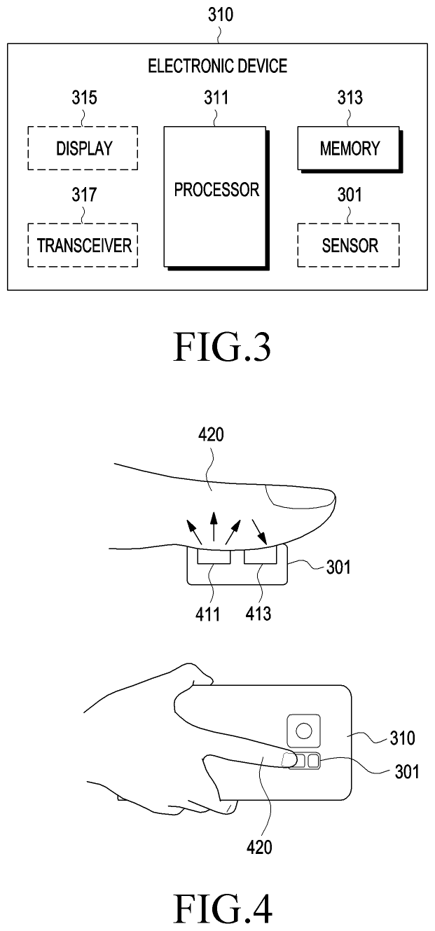

FIG. 3 is a block view of an electronic device according to an embodiment of the disclosure.

Referring to FIG. 3, the electronic device 310 may include a processor 311 and a memory 313. The electronic device 310 may further include at least one of a sensor 301, a display 315 or a transceiver 317. The processor 311, the memory 313, the sensor 301, the display 315 and the transceiver 317 may be electrically or operatively connected to each other to exchange signals (e.g., instruction or data) therebetween.

The sensor 301 may generate and output a sensing signal (e.g., PPG signal) related to the user's breathing. The sensing signal may be generated in response to user input (e.g., a user's action of placing a finger on the sensor 301 side).

The sensing operation of the sensor 301 may be triggered by user input to the sensor 301. For example, when the user's second body part (e.g., finger) is in contact with the sensor 301 or is located near the sensor 301, a sensing signal may be output from the sensor 301 and transmitted to the processor 311. The second body part may be an oxygen delivery point (e.g., tissue such as the skin of a finger).

The processor 311 may receive a sensing signal related to the user's breathing from the sensor 301.

The processor 311 may obtain the first pattern and the second pattern, based on the sensing signal received from the sensor 301. For example, the first pattern may be a pattern related to a change in breathing of a user over time. The second pattern may be a pattern related to a change in saturation of peripheral oxygen of the user over time.

The processor 311 may obtain a first pattern (e.g., a respiration pattern) based on a sensing signal related to the user's breathing output from the sensor 301. For example, when the sensor 301 is a multiple-wavelength PPG type, a user's breathing pattern may be identified from the PPG signal output from the sensor 301.

The processor 311 may obtain a second pattern (e.g., a saturation of peripheral oxygen pattern), based on a sensing signal related to the user's breath output from the sensor 301. For example, when the sensor 301 is a multiple-wavelength PPG type, the saturation of peripheral oxygen pattern of the user may be identified from PPG signals of different wavelengths output from the sensor 301.

The processor 311 may determine an oxygen delivery time, based on a comparison (e.g., pattern matching) between the first pattern (e.g., respiration pattern) and the second pattern (e.g., saturation of peripheral oxygen pattern).

The oxygen delivery time may indicate the time required for oxygen to be delivered from the first body part (e.g., the lungs) of the user to the second body part (oxygen delivery point, e.g., a finger) of the user through the blood.

The oxygen delivery time may be determined (measured) by the time difference between the first pattern (e.g., respiration pattern) and the second pattern (e.g., saturation of peripheral oxygen pattern). The first pattern and the second pattern may be measured at one oxygen transfer point (e.g., finger). The first pattern and the second pattern may be obtained by a single sensor 301 located at one oxygen delivery point.

In various embodiments, the processor 311 may identify the first section of the first pattern and the second section of the second pattern by comparing the first pattern and the second pattern related to the user's breath. The processor 311 may identify a time-delay value according to a difference between a start time point of the first section and a start time point of the second section. The processor 311 may determine an oxygen delivery time corresponding to the identified time-delay value.

In various embodiments, the processor 311 may determine information related to a cardiovascular system parameter (e.g., blood pressure information), based on the oxygen delivery time and display the same on the screen of the display 315. The display 315 may display at least a part of information related to oxygen delivery time or information related to cardiovascular system parameters (e.g., blood pressure information) calculated therefrom, under the control of the processor 311.

The memory 313 may store instructions for the operation of the processor 311 and cause the processor 311 to perform the operation of receiving the sensing signal, the operation of obtaining the patterns, and the operation of determining the oxygen delivery time when the instructions are executed.

In various embodiments, synchronized patterns may be obtained as a plurality of patterns (e.g., breathing patterns and blood oxygen saturation patterns) related to a user's breathing are obtained using the sensor 301 as a single source. The plurality of patterns, obtained based on the sensing signal from the same sensor 301, may have the same time stamp. A plurality of patterns obtained based on the same sensing signal may be registered (stored) with the same time stamp.

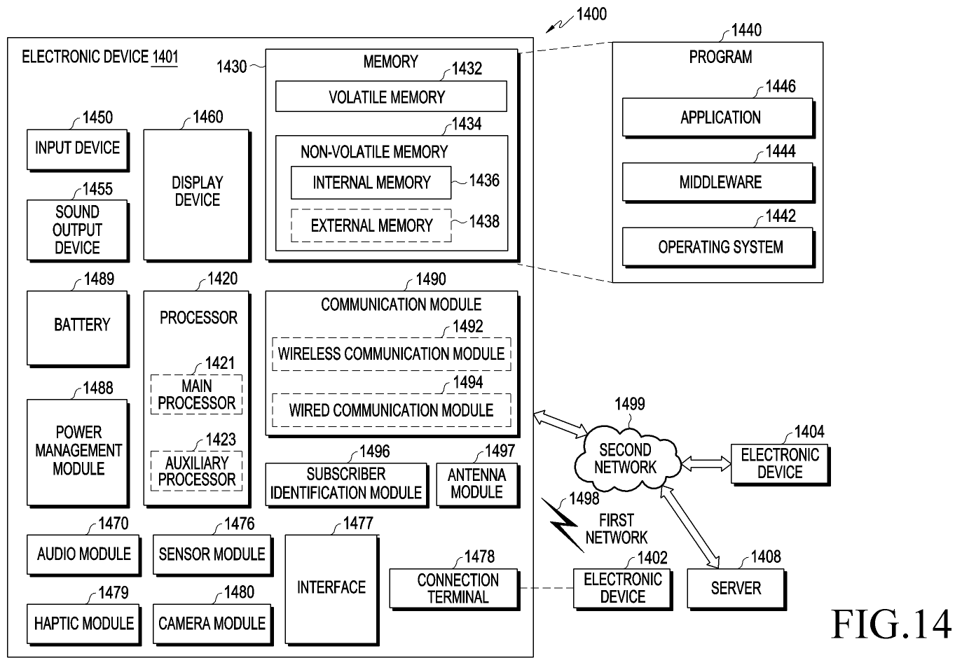

The electronic device 310 may provide a health care service using the oxygen delivery time measured through the sensor 301. The electronic device 310 may interwork with an external server (e.g., a server 1408 of FIG. 14) through the transceiver 317 for healthcare services. For example, the electronic device 310 may measure the oxygen delivery time of the user using the sensor 301 and provide information about the oxygen delivery time to a server through the transceiver 317 (e.g., a server 1408 of FIG. 14). The server (e.g., a server 1408 of FIG. 14) may estimate a user's cardiovascular system parameters (e.g., blood pressure, blood viscosity, and blood vessel state), based on information about the received oxygen delivery time, and may diagnose, monitor, and manage the user's state of health using the cardiovascular system parameters. The server (e.g., the server 1408 of FIG. 14) may provide information about the user's state of health (e.g., a change in blood pressure of the user, a diagnosis result, a monitoring result) to the electronic device 310 through the transceiver 317.

The transceiver 317 may transmit information about the oxygen delivery time of the user to the server (e.g., the server 1408 in FIG. 14) under the control of the processor 311, or may receive information about the user's state of health from the server (e.g., the server 1408 in FIG. 14).

In various embodiments, the electronic device 310 may include some or all of the components of the electronic device 1401 illustrated in FIG. 14 described below. For example, the processor 311, the memory 313, the sensor 301, the display 315, and the transceiver 317 of the electronic device 310 may respectively correspond to a processor 1420, a memory 1430, a sensor module 1476, a display device 1460, and a communication module 1490 of the electronic device 1401 shown in FIG. 14.

FIG. 4 is a view illustrating the use of an electronic device according to an embodiment of the disclosure.

In various embodiments, the electronic device 310 may obtain a plurality of patterns related to a user's breathing, for example, a blood pattern and a saturation of peripheral oxygen pattern, based on a sensing signal output from the sensor 301.

The saturation of peripheral oxygen may be measured by surgical or non-surgical approaches. In the case of a surgical approach, there may be inconvenience and hassle for the user, such as requiring a painful surgical measurement operation, requiring a professional medical device with low availability, and requiring insertion of a marker material.

In the case of a non-surgical approach, a simple and user-friendly measurement method may be applied. However, in this case, a separate additional sensor (e.g., an additional airflow sensor or a breathing sensor other than the PPG sensor) for measuring the user's breathing may be required. In addition, additional respiratory measurement procedures, including user actions, may be required in order to utilize the additional sensor.

Various embodiments may provide a non-surgical approach that may be measured in a simple and user-friendly manner, instead of a surgical approach that causes user discomfort and hassle.

Various embodiments provide a non-surgical approach, but it is possible to obtain a plurality of patterns (e.g., a respiration pattern and a saturation of peripheral oxygen pattern) necessary for determination of the oxygen delivery time simultaneously through a simple user input (e.g., a method in which a user places a finger on the sensor 301 side included in the electronic device 310) with respect to the sensor 301 (e.g. multiple-wavelength PPG sensor), without using additional sensors (e.g. airflow sensors or breathing sensors).

Referring to FIG. 4, a sensing operation may be triggered to generate a sensing signal in response to a user input to the sensor 301. As the user's second body part (e.g., finger 420) contacts the sensor 301 or is located near the sensor 301, a sensing operation of the sensor 301 may be triggered to generate a sensing signal.

The sensor 301 may be included in the electronic device 310 or may be used in connection with the electronic device 310. For example, the sensor 301 may be embedded in the electronic device 310 as shown. As another example, the sensor 301 may be configured to be detachably attached to the electronic device 310 (e.g., a form connected by a connector or an inserted module form).

In various embodiments, the sensor 301 may be a multiple-wavelength PPG sensor. The sensing signal output from the sensor 301 may include PPG signals of different wavelengths (e.g., a red PPG signal and an infrared PPG signal).

For example, the sensor 301 may include an illumination module 411 emitting light of multiple wavelengths (e.g., two or more wavelengths), and a photodiode 413 for outputting a sensing signal according to a user's input (e.g., contact or approach) to the sensor 301.

In order to obtain the patterns necessary for the measurement of the oxygen delivery time, the sensor 301 may emit light of multiple wavelengths (e.g., infrared light and red light). The electronic device 310 may obtain a sensing signal and patterns based on the sensing signal in the state in which multiple wavelengths of light are emitted.

In the state in which the multiple wavelengths of light are emitted through the illumination module 411, when the user's finger 420 is in contact with the sensor 301 or a part of the electronic device 310 in which the sensor 301 is configured, or is located near the sensor 301, a sensing signal may be generated and output through the sensor 301.

As an example, the illumination module 411 may include an infrared light source and a red light source.

The sensing signal on the user finger 420 may be measured through the sensor 301. The sensing signal may include a PPG signal of a first wavelength (e.g., a red PPG signal) and a PPG signal of a second wavelength (e.g., an infrared PPG signal).

The electronic device 310 may receive a sensing signal (e.g., a PPG signal) on the user's finger 420, and may obtain a plurality of patterns related to the user's breathing, for example, the user's respiration pattern and the user's saturation of peripheral oxygen pattern, based on the sensing signal.

The method of obtaining the respiration pattern and the saturation of peripheral oxygen pattern from the PPG signal is described below.

The electronic device 310 may obtain a user's respiration pattern from a PPG signal (e.g., a red PPG signal or an infrared PPG signal). For example, respiratory values mapped to the PPG signal level may be stored in advance in the form of a lookup table. After obtaining the PPG signal output from the sensor 301, the electronic device 310 may extract a respiratory value corresponding to the obtained PPG signal level obtained from the lookup table. The electronic device 310 may collect the extracted discrete respiratory values, obtain a respiratory pattern representing a change in the magnitude of the respiratory values over time, and use the respiratory pattern to determine an oxygen delivery time.

The electronic device 310 may obtain a user's saturation of peripheral oxygen pattern from the PPG signal.

The saturation of peripheral oxygen may refer to the ratio of oxygenated hemoglobin (oxygen-containing hemoglobin) and blood hemoglobin (oxygenated and non-oxygenated hemoglobin). In order to obtain the saturation of peripheral oxygen pattern, the sensor 301 may emit light of a first wavelength and light of a second wavelength (e.g., infrared light and red light), having different light transmittances for oxygenated hemoglobin and non-oxygenated hemoglobin, through the illumination module 411. The sensor 301 may output a PPG signal through the photodiode 413 in response to user input in the state in which multiple wavelengths of light are emitted.

Multiple wavelengths (e.g., wavelengths respectively corresponding to two or more light-emitting diodes (LEDs)) may be required in order to obtain a blood oxygen saturation pattern. For example, a pair of wavelengths including infrared light (long wavelength) and red light (short wavelength) may be used. Alternatively, any other pair of light wavelengths (a pair of light sources having different wavelengths) may be used.

As an example, oxygenated hemoglobin may absorb more infrared (longer-wavelength) light, allowing more red light to pass through, compared to non-oxygenated hemoglobin. Non-oxygenated hemoglobin may pass more infrared (longer-wavelength) light, allowing more red (shorter-wavelength) light to be absorbed, compared to oxygenated hemoglobin. The amount of light that is not absorbed may be measured, and separate normalized signals may be generated for each wavelength. Since the actual amount of arterial blood present in a tissue (e.g., the skin of the finger 420) increases with each heart rate, the signals for each wavelength may fluctuate over time.

The influence of other tissues may be corrected in order to generate a continuous signal for pulsatile arterial blood by subtracting the minimum transmitted light from the transmitted light of each wavelength. The continuous signal may be a PPG signal. The ratio of the red (shorter-wavelength) light quantity measurement value to the infrared (longer-wavelength) light quantity measurement value may be calculated from the PPG signal. The measured value represents the ratio of oxygenated hemoglobin to non-oxygenated hemoglobin, and may be expressed as in Equation 1.

$$R = \frac{{HbO}_{2}}{{HbO}_{2} + Hb} \qquad Equation 1$$

Here, R may be defined as a ratio, HbO₂ as oxygenated hemoglobin, and Hb as non-oxygenated hemoglobin.

The electronic device 310 may measure oxygen saturation in the blood, based on the ratio calculated using Equation 1. For example, the ratio may be converted into a saturation of peripheral oxygen value using a lookup table, based on the Beer-Lambert rule (the law stating that the fraction of absorbed light is proportional to the concentration of a substance).

A lookup table indicating the mapping relationship between the ratio of oxygenated hemoglobin to non-oxygenated hemoglobin-blood oxygen saturation may be stored in advance. After obtaining the ratio of oxygenated hemoglobin to non-oxygenated hemoglobin from the PPG signal, the electronic device 310 may extract a saturation of peripheral oxygen value corresponding to the ratio from the lookup table. The electronic device 310 may collect the extracted discrete saturation of peripheral oxygen values, and obtain a saturation of peripheral oxygen pattern representing a change in the saturation of peripheral oxygen values over time, and may use the saturation of peripheral oxygen pattern in order to determine the oxygen delivery time.

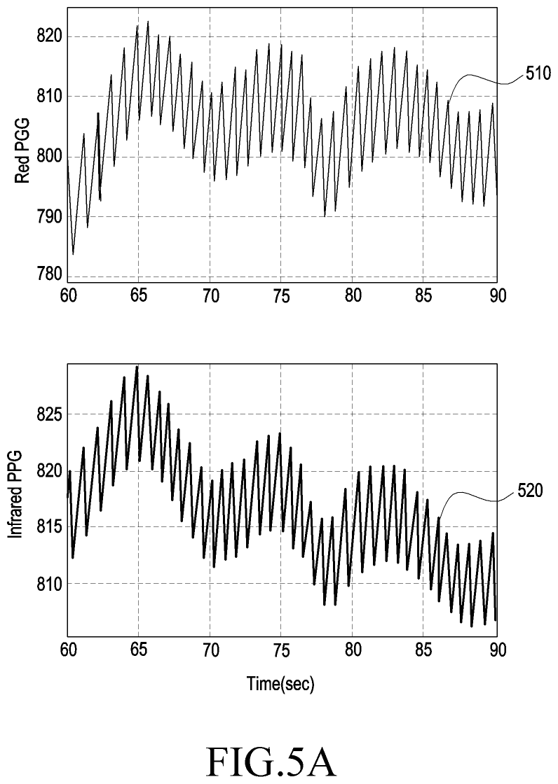

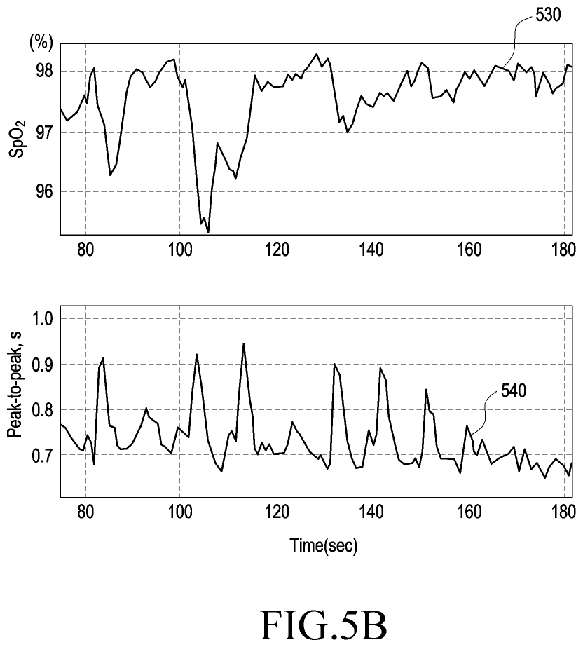

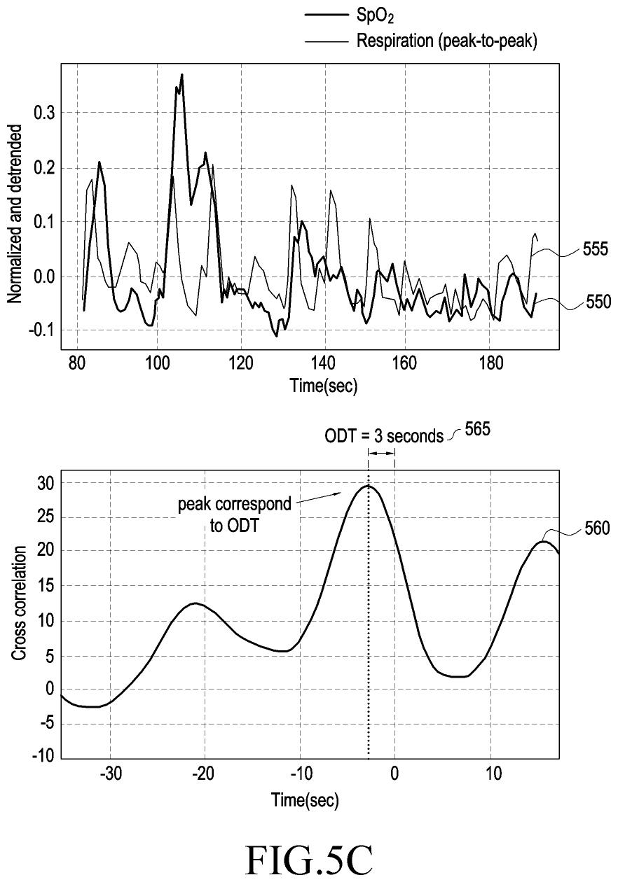

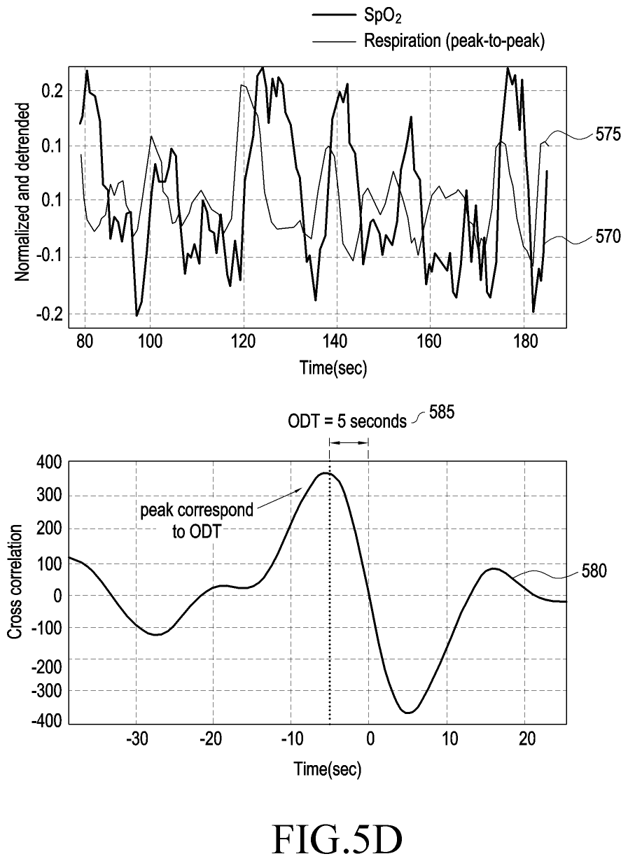

FIG. 5A is a graph illustrating an operation of an electronic device according to an embodiment of the disclosure, FIG. 5B is a graph illustrating an operation of an electronic device according to an embodiment of the disclosure, FIG. 5C is a graph illustrating an operation of an electronic device according to an embodiment of the disclosure, and FIG. 5D is a graph illustrating an operation of an electronic device according to an embodiment of the disclosure.

FIG. 5A illustrates a sensing signal output from the sensor 301. The sensing signal may include a first-wavelength PPG signal 510 (e.g., a red PPG signal) and a second-wavelength PPG signal 520 (e.g., an infrared PPG signal). For example, each of the PPG signal 510 of the first wavelength and the PPG signal 520 of the second wavelength may be output current of the photodiode 413 included in the sensor 301 of FIG. 4 or a signal corresponding thereto.

FIG. 5B is a graph illustrating a blood oxygen saturation pattern 530 representing a change in the saturation of peripheral oxygen of a user over time and a respiration pattern 540 representing a change in the breathing of a user over time.

The electronic device 310 may measure the saturation of peripheral oxygen pattern 530 and the respiration pattern 540 using a sensing signal. For example, the respiration pattern 540 according to the peak-to-peak interval (PPI) of the first wavelength PPG signal (510 in FIG. 5A) or the second wavelength PPG signal (520 in FIG. 5A) sensed on the finger may be measured. For example, the saturation of peripheral oxygen pattern 530 may be measured based on the PPG signal 510 of the first wavelength and the PPG signal 520 of the second wavelength, sensed on the finger.

FIG. 5C is a graph illustrating a cross correlation between a respiration pattern and a blood oxygen saturation pattern.

The respiration pattern (e.g., 540 in FIG. 5B) and the saturation of peripheral oxygen pattern (e.g., 530 in FIG. 5B) may be signal-processed (e.g., normalized and detrended) for mutual comparison.

Reference numerals 550 and 555 denote normalized and trend-removed saturation of peripheral oxygen pattern signals and respiratory pattern signals, respectively. Reference numeral 560 denotes a graph showing the correlation between the saturation of peripheral oxygen pattern signal 550 and the respiration pattern signal 555. The peak of the signal 560, indicating the correlation between the two pattern signals 550 and 555, may define the point having the maximum correlation between the respiration pattern and the saturation of peripheral oxygen pattern.

As illustrated in FIG. 5C, the oxygen delivery time 565 may correspond to the peak of the signal 560, indicating the correlation between the two pattern signals 550 and 555. The oxygen delivery time 565 may be determined (measured) based on the peak. The oxygen delivery time 565 may be determined as a value corresponding to a section from a reference time point (origin) to the time point where the correlation between the saturation of peripheral oxygen pattern and the respiration pattern is highest. In the example of FIG. 5C, the oxygen delivery time 565 may be determined as 3 seconds.

FIG. 5D is a graph showing another example of a cross-correlation between two patterns.

Reference numerals 570 and 575 denote normalized and trend-removed saturation of peripheral oxygen pattern signals and respiration pattern signals, respectively. Reference numeral 580 denotes a cross-correlation between the saturation of peripheral oxygen pattern signal 570 and the respiration pattern signal 575. The peak of reference numeral 580 may correspond to the oxygen transfer time 585, and the oxygen transfer time 585 may be determined based on the peak. In the example of FIG. 5D, the oxygen delivery time or oxygen transfer 585 may be determined (measured) as 5 seconds.

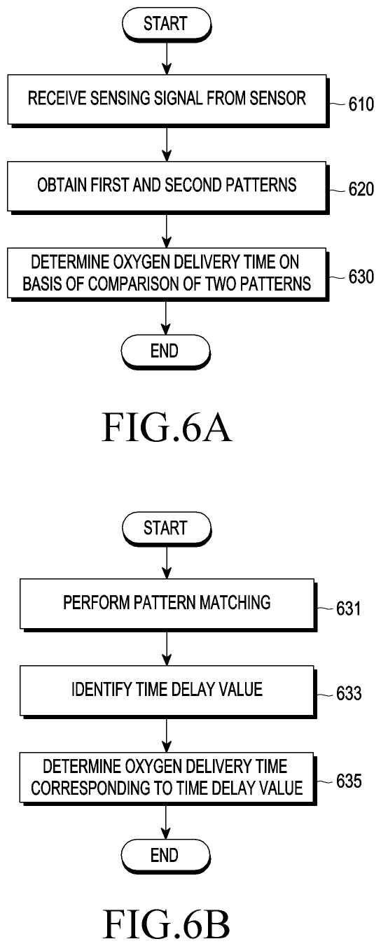

FIG. 6A is a flowchart illustrating an operation of obtaining a vital sign according to an embodiment of the disclosure, and FIG. 6B is a flowchart illustrating an operation of obtaining a vital sign according to an embodiment of the disclosure.

Referring to FIGS. 6A and 6B, at least some of the vital sign acquisition operations may be performed by the electronic device 310 or at least one processor 311. For convenience, hereinafter, it is assumed that the operation is performed by the electronic device 310.

At operation 610, the electronic device 310 may receive a sensing signal from the sensor 301 connected to the electronic device 310. The electronic device 310 may receive a sensing signal when the user's second body part (oxygen delivery point, e.g., a finger) contacts the sensor 301 or is located near the sensor 301.

A sensing operation may be triggered to generate a sensing signal in response to user input to the sensor 301. For example, a sensing operation may be triggered in response to user input, performed using a finger, on the sensor 301.

For example, the electronic device 310 may execute a specific application (e.g., a healthcare application) and display a screen requesting user input for a sensing operation through the application. When the screen is displayed, the state of the sensor 301 may be switched (e.g., from a sleep state to a standby state waiting for user input). When the screen is displayed, an indication for requesting user input to the sensor 301 (e.g., a notification message, sound, light emission of the sensor 301, etc.) may be output therewith.

At operation 620, the electronic device 310 may obtain a first pattern related to the breathing signal and a second pattern related to the peripheral oxygen saturation signal, based on the sensing signal. For example, a first pattern and a second pattern related to breathing of the user may be generated from a sensing signal, sensed for a certain time (e.g., 2 seconds), on a user's finger.

At operation 630, the electronic device 310 may determine the oxygen delivery time, based on the comparison between the first pattern and the second pattern. The oxygen delivery time may be determined based on the time difference between the first pattern and the second pattern.

The oxygen delivery time may indicate the time taken for oxygen to be delivered from the first body part of the user to the second body part of the user through the blood. As an example, the first body part may correspond to the lungs, and the second body part may correspond to the finger.

In various embodiments, operation 630 may include operation 631, operation 633 and operation 635 of FIG. 6B.

At operation 631, the electronic device 310 may compare the first pattern and the second pattern to identify the first section of the first pattern and the second section of the second pattern (e.g., 231 and 241 of FIG. 2C), which are pattern-matched. At operation 633, the electronic device 310 may identify a time-delay value according to the difference between the identified start time of the first section and the start time of the second section. At operation 635, the electronic device 310 may determine an oxygen delivery time corresponding to the identified time-delay value.

The electronic device 310 may interwork with an external server (e.g., the server 1408 of FIG. 14) for the measurement of oxygen delivery time and a healthcare service using the oxygen delivery time. For example, the electronic device 310 may interwork with an external server (e.g., the server 1408 of FIG. 14) that provides service related to a specific application (e.g., a healthcare application).

The electronic device 310 may transmit information about an oxygen delivery time to an external server (e.g., the server 1408 in FIG. 14), receive information about the cardiovascular system parameters, based on the oxygen delivery time, and display the screen.

The electronic device 310 may transmit and register (store) the oxygen delivery time or patterns used for determining the oxygen delivery time to an external server (e.g., the server 1408 of FIG. 14) along with a time stamp. The oxygen delivery time may be included in a database on the electronic device 310 that stores information about the user's state of health and may be used to provide healthcare service for the user (e.g., monitoring, diagnosis, and management of the user's state of health). For example, information about a user's oxygen delivery time provided from the electronic device 310 may be accumulated for a certain period (e.g., by date and month), and may be used to provide a healthcare service to the user.

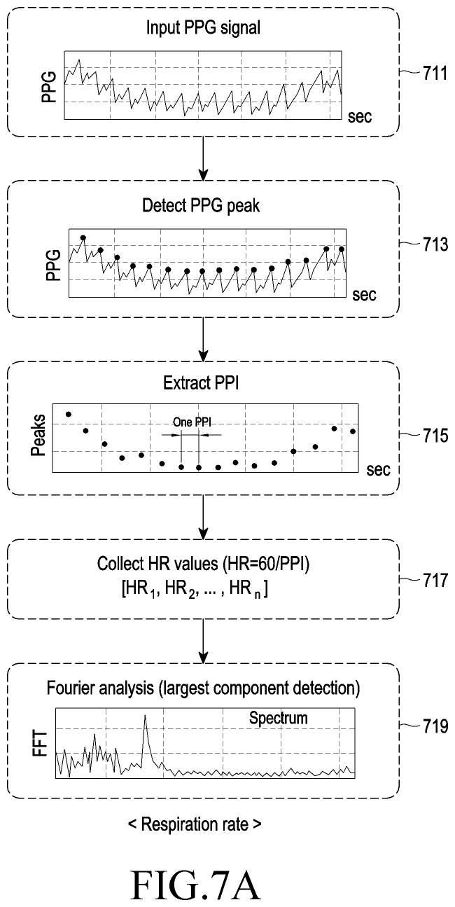

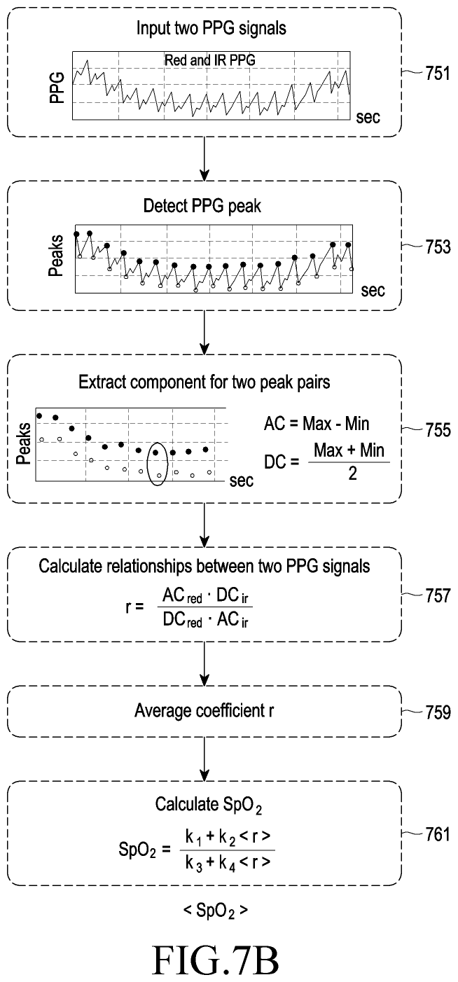

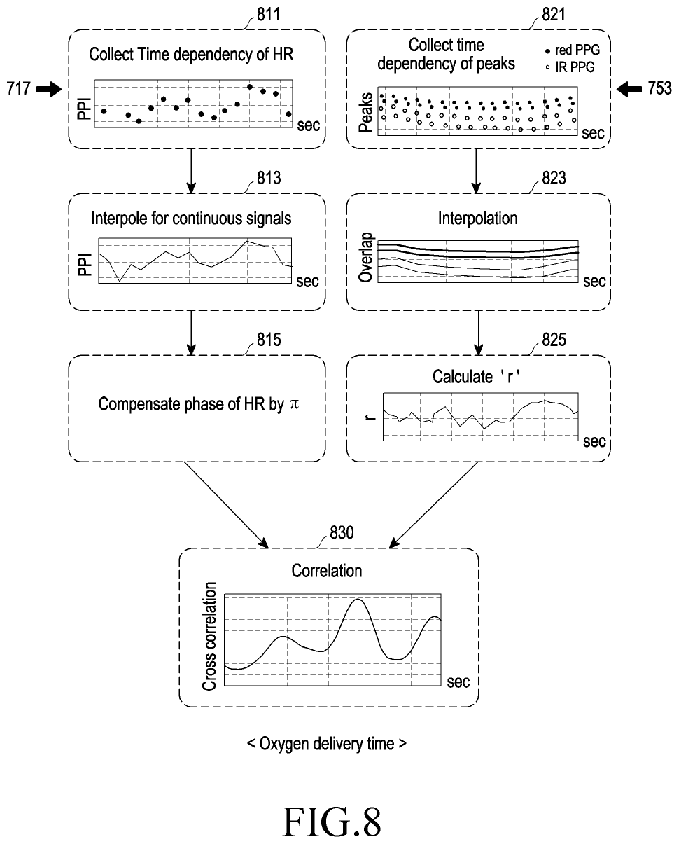

FIG. 7A is a graph illustrating a vital-sign acquisition algorithm according to an embodiment of the disclosure, FIG. 7B is a graph illustrating a vital-sign acquisition algorithm according to an embodiment of the disclosure, and FIG. 8 is a graph illustrating a vital-sign acquisition algorithm according to an embodiment of the disclosure.

FIG. 7A illustrates a known operation for measuring respiration values, and FIG. 7B illustrates a known operation for measuring a saturation of peripheral oxygen value. FIG. 8 illustrates an operation of determining an oxygen delivery time according to various embodiments.

FIG. 7A is for explaining a known respiration rate measurement method, and illustrates a respiration rate (bpm) detection technique, based on a PPG signal.

Referring to FIG. 7A, the respiration rate may be measured via the operation of inputting (711) the PPG signal, detecting (713) the peaks of the PPG signal, extracting (715) the peak-to-peak interval (PPI), collecting (717) heart rate (HR: instant heart rate (bpm)) values, and performing Fourier analysis (detection of the maximum component) 719.

Each heart rate value, collected via operation 717, may indicate a respiration rate (respiration value). Referring to operation 717, it may be seen that only the size sequence of the PPI values is required in order to measure the respiration rate. In consideration thereof, in various embodiments, a respiration pattern indicating a change in period of breathing may be obtained, and the respiration pattern may be used to measure oxygen delivery time. For example, the respiration pattern may correspond to the size sequence of the PPI values obtained via operation 717.

FIG. 7B illustrates a method for measuring saturation of peripheral oxygen at a known time point, and illustrates a technique for measuring saturation of peripheral oxygen (SpO₂, %), based on a red PPG signal and an infrared PPG signal.

Referring to FIG. 7B, the saturation of peripheral oxygen may be measured via the operation of inputting (751) a red PPG signal and an infrared PPG signal, detecting (753) PPG peaks, extracting (755) an alternative and fixed component for a pair of two peaks, calculating (757) the correlation coefficient r between the infrared PPG signal and the red PPG signal for one time point, averaging (759) the coefficient r, and calculating (761) the saturation of peripheral oxygen value.

Referring to operation 755, it may be seen that a known peripheral oxygen saturation measurement method aims to measure an average of peripheral oxygen saturation level. Referring to operation 759, it may be seen that averaging in a known corresponding scheme ignores all time dependencies. In various embodiments, a saturation of peripheral oxygen pattern reflecting a time-dependent property, for example, a pattern representing a time-amplitude change in saturation of peripheral oxygen, may be obtained and used to measure oxygen delivery time. As an example, the saturation of peripheral oxygen pattern may be generated based on the PPG peak values obtained via operation 753 before time dependency is removed.

FIG. 8 is for describing an algorithm for measuring an oxygen delivery time applicable to various embodiments. In various embodiments, a technique for measuring a saturation of peripheral oxygen value at one time point as shown in FIG. 7B may be extended to pattern monitoring and used for measuring a saturation of peripheral oxygen pattern. The respiration rate measurement technique shown in FIG. 7A may be extended to pattern monitoring and used for measuring respiration patterns.

The respiration pattern and the saturation of peripheral oxygen pattern may be detected at the same oxygen delivery point (e.g., a user's finger).

In various embodiments, the operation of determining the oxygen delivery time may include at least some of the operations of 811 to 830.

An overall size-phase time-dependent evaluation of the PPI signal may be required for respiration pattern detection. At operation 811, a time dependency of heart rate values may be collected. To this end, the heart rate values obtained via operation 717 of FIG. 7A may be provided. At operation 813, interpolation to obtain a continuous signal may be performed. At operation 815, phase compensation of the heart rate signal may be performed by 7C.

A respiration pattern may be obtained via operation 815, which compensates for the phase of the heart rate signal. With regard to operation 815, since heart rate is low at a high oxygen level in the lungs, instantaneous heart rate modulation may be achieved and heart rate signals may be obtained by converting respiratory signals in anti-phase. This phase relationship may also be applied to the opposite case.

In order to detect the saturation of peripheral oxygen pattern, it may be necessary to evaluate the overall size-phase time dependence of alternating-current (AC) and direct-current (DC) components of the red PPG signal and the infrared PPG signal. At operation 821, the time dependencies of peaks of the red PPG signal and the infrared PPG signal may be collected. To this end, the PPG peak values obtained via operation 753 of FIG. 7B may be provided.

At operation 823, interpolation for successive envelopes (max and min) of the red and infrared PPG signals may be performed. At operation 825, a continuous signal of time-dependent coefficient r may be generated. R at each time point may be a value representing the ratio of oxygenated hemoglobin to non-oxygenated hemoglobin. The saturation of peripheral oxygen pattern may be obtained from the continuous signal.

At operation 830, a correlation between the two patterns may be measured to detect a time difference between the two patterns (respiration pattern and saturation of peripheral oxygen pattern). For the measurement of the correlation, a respiration pattern obtained via operation 815 and a saturation of peripheral oxygen pattern obtained via operation 825 may be used. The oxygen transfer time may be determined using a time difference detected from the correlation between the two patterns.

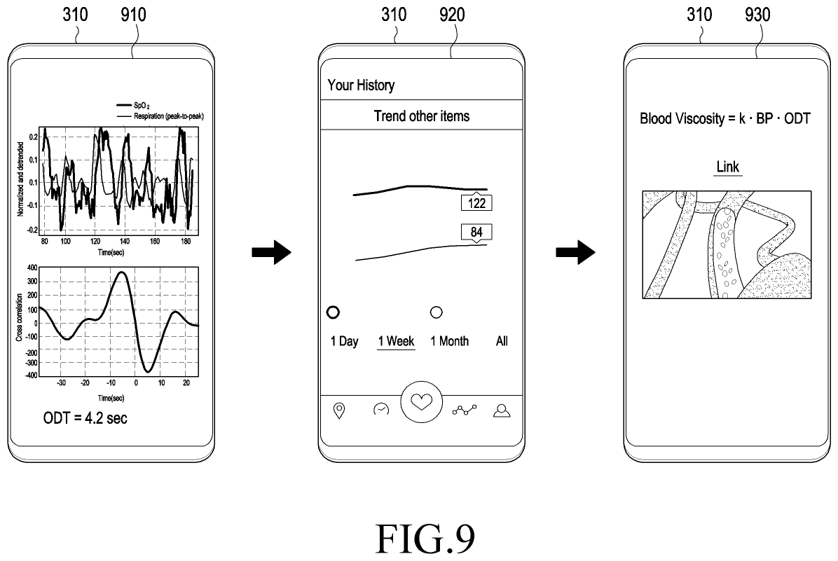

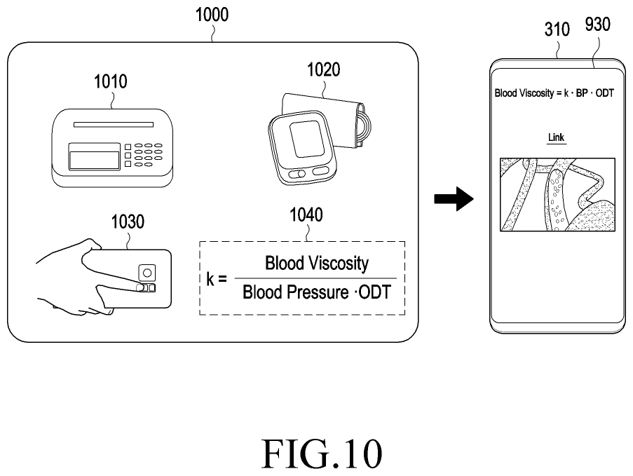

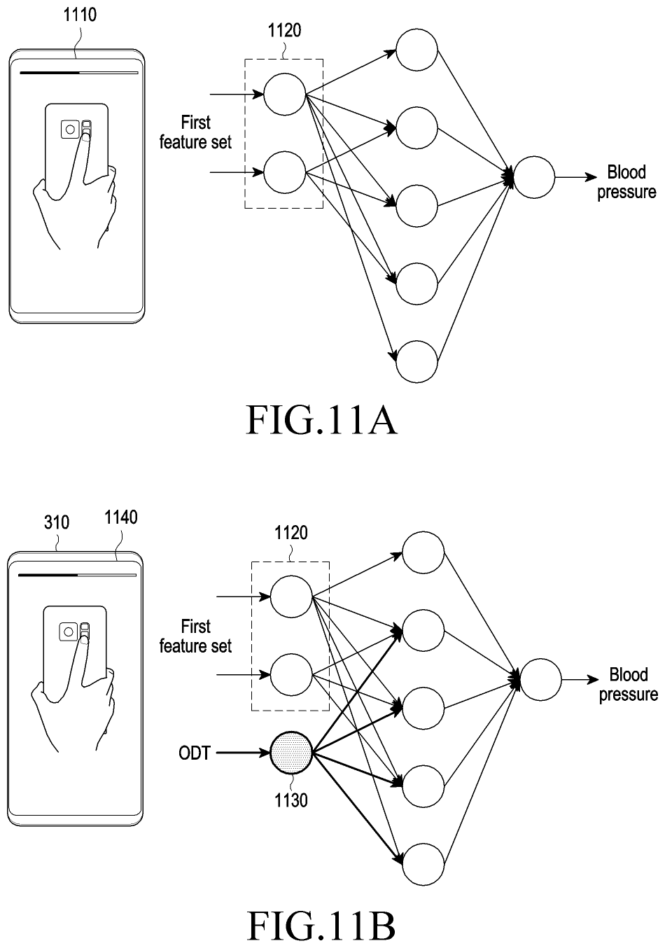

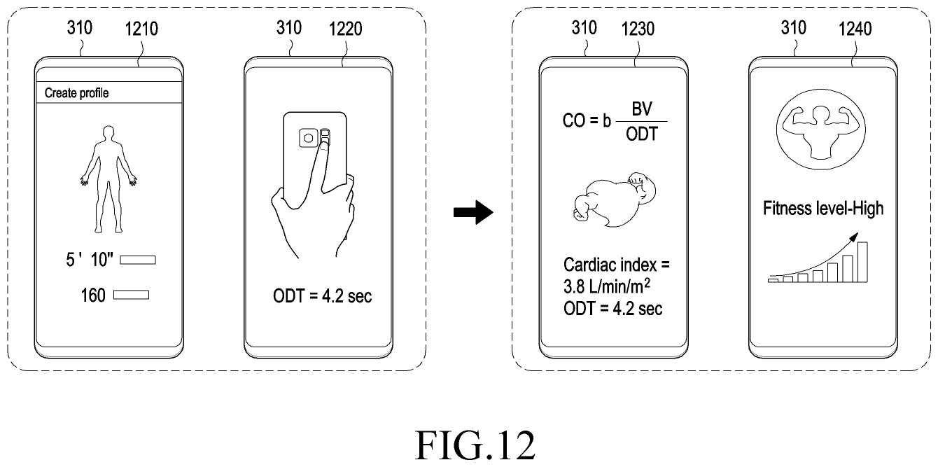

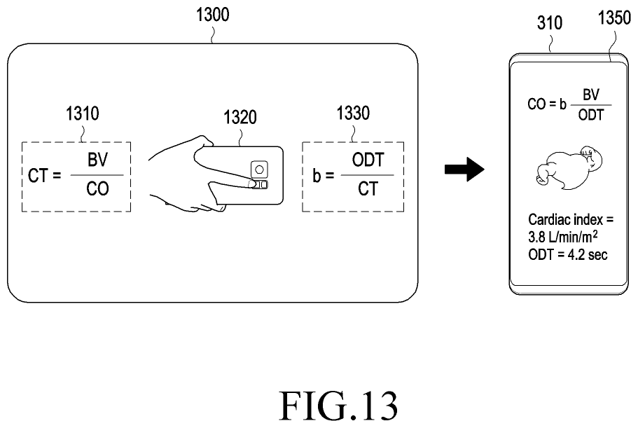

FIG. 9 is a view illustrating a method of using a vital-sign parameter obtained according to an embodiment of the disclosure, FIG. 10 is a view illustrating a method of using a vital-sign parameter obtained according to an embodiment of the disclosure, FIG. 11A is a view illustrating a method of using a vital-sign parameter obtained according to an embodiment of the disclosure, FIG. 11B is a view illustrating a method of using a vital-sign parameter obtained according to an embodiment of the disclosure, FIG. 12 is a view illustrating a method of using a vital-sign parameter obtained according to an embodiment of the disclosure, and FIG. 13 is a view illustrating a method of using a vital-sign parameter obtained according to an embodiment of the disclosure. For example, at least some of the operations or screens described through the drawings may be provided by an application (e.g., a healthcare application) executed on the electronic device 310. To this end, the application may interwork with an external server (e.g., the server 1408 in FIG. 14).

Precise measurement of various cardiovascular system parameters (e.g., blood pressure, blood viscosity, vascular condition) illustrated in FIGS. 9 to 13 may be possible using an oxygen transfer time. Accordingly, the accuracy of the healthcare service, and user satisfaction therewith, may be improved.

FIG. 9 is an example of estimating blood viscosity using an oxygen delivery time, which is a vital sign.

Reference numeral 910 illustrates a screen of an electronic device 310 that measures the oxygen delivery time. Reference numeral 920 illustrates a screen of the electronic device 310 that measures blood pressure using the oxygen delivery time measured through the screen indicated by reference numeral 910.

Reference numeral 930 illustrates a screen of the electronic device 310 showing measurement of a blood viscosity trend using the blood pressure measured through the screen of reference numeral 920. The blood viscosity may be measured by the formula of “Blood Viscosity=k·BP·ODT”. Here, Blood Viscosity may be defined as blood viscosity, k as an index, BP as a heart rate, and ODT as an oxygen delivery time.

The index k may be personalized by preliminary calibration (see the description of FIG. 10 below). When calibration is performed, the oxygen delivery time can more accurately show the actual value of the blood viscosity. In the absence of correction, the blood viscosity trend may be identified.

The blood viscosity may be used, for example, for healthcare services that detect cardiovascular event risk, cognitive decline, diabetes complications, and the like.

FIG. 10 is an example of estimating blood viscosity using an oxygen delivery time, which is a vital sign.

Reference numeral 1000 relates to a calibration operation of the index k. The screen of reference numeral 930 relates to the measurement of the blood viscosity trend.

The index k may be personal for each user. The index k may be personalized by preliminary calibration. When calibration is performed, the oxygen delivery time can more accurately show the actual value of blood viscosity. In the absence of correction, the trend of blood viscosity changes may be identified.

As an example, the operation (1000) of calibrating the index k may include an operation (1010) of measuring blood viscosity by a surgical means in a medical clinic, an operation (1020) of measuring blood pressure, and an operation (1030) of measuring oxygen delivery time and re-measuring the index k (at operation 1040).

The blood viscosity trend measurement is performed by applying the index k, measured as described above, and may be displayed on the screen as indicated by reference numeral 930.

FIGS. 11A and 11B are examples of performing improved blood pressure estimation using a vital sign, that is, oxygen delivery time.

FIG. 11A illustrates the case in which blood pressure is measured in consideration only of the first feature set 1120 for PPG-type features.

A screen requesting user input for a sensor (e.g., a PPG sensor) may be displayed on an electronic device as indicated by reference numeral 1110, and the first feature set 1120 (e.g., heart rate, saturation of peripheral oxygen) may be identified according to the user input. The blood pressure may be measured based on the first feature set 1120, and the measured blood pressure information may be provided on a screen.

FIG. 11B illustrates the case in which the blood pressure is measured in consideration of the first feature set 1120 and the oxygen delivery time 1130 for PPG-type features. A screen requesting user input to the sensor 301 may be displayed on the electronic device 310, as shown at 1140. Both the first feature set 1120 (e.g., heart rate, saturation of peripheral oxygen) and the oxygen delivery time 1130 may be identified depending on the user input to the sensor 301. The blood pressure may be measured based on the first feature set 1120 and the oxygen delivery time 1130, and thus the measured blood pressure information may be provided to the screen.

Compared to the case of FIG. 11A, in which the blood pressure is measured in consideration only of the first feature set 1120, when measuring the blood pressure in consideration of the first feature set 1120 and the oxygen delivery time 1130, as shown in FIG. 11B, the accuracy of blood pressure estimation may be increased. When using the oxygen delivery time 1130, a blood flow rate that is opposite to the oxygen delivery time may be known, thereby increasing the accuracy of blood pressure estimation.

FIG. 12 is an example of performing cardiac output/fitness level estimation using an oxygen delivery time, which is a vital sign.

Reference numeral 1210 illustrates a screen of the electronic device 310 for receiving a body blood volume. The body blood volume may be input based on personal parameters (e.g., age, gender, weight, height) from a database that stores information about the user's state of health.

Reference numeral 1220 illustrates a screen of the electronic device 310 for measuring the oxygen delivery time. Reference numeral 1230 illustrates a screen of the electronic device 310 for measuring the cardiac output/index. Reference numeral 1240 illustrates a screen of the electronic device 310 for estimating the fitness level trend.

FIG. 13 is an example of performing cardiac output/fitness level estimation using an oxygen delivery time, which is a vital sign.

Reference numeral 1300 relates to a calibration operation of the index b. Reference numeral 1350 relates to cardiac output measurement.

Index b may be personal for each user. Index b may be personalized (or customized) by preliminary calibration. When calibration is performed, the oxygen delivery time can accurately show the actual value of cardiac output. In the absence of correction, the trend of cardiac output changes may be identified.

For example, the operation (1300) of calibrating the index b may include an operation (1310) of measuring a blood circulation time, an operation (1320) of measuring an oxygen delivery time, and an operation (1330) of measuring an index b.

After the cardiac output is measured by applying the index b measured as described above, the cardiac output may be provided on a screen of the electronic device 310, such as 1350.

FIG. 14 is a block diagram illustrating an electronic device 1401 in a network environment 1400 according to various embodiments.

Referring to FIG. 14, the electronic device 1401 in the network environment 1400 may communicate with an electronic device 1402 via a first network 1498 (e.g., a short-range wireless communication network), or an electronic device 1404 or a server 1408 via a second network 1499 (e.g., a long-range wireless communication network). According to an embodiment, the electronic device 1401 may communicate with the electronic device 1404 via the server 1408. According to an embodiment, the electronic device 1401 may include a processor 1420, memory 1430, an input device 1450, a sound output device 1455, a display device 1460, an audio module 1470, a sensor module 1476, an interface 1477, a haptic module 1479, a camera module 1480, a power management module 1488, a battery 1489, a communication module 1490, a subscriber identification module (SIM) 1496, or an antenna module 1497. In some embodiments, at least one (e.g., the display device 1460 or the camera module 1480) of the components may be omitted from the electronic device 1401, or one or more other components may be added in the electronic device 1401. In some embodiments, some of the components may be implemented as single integrated circuitry. For example, the sensor module 1476 (e.g., a fingerprint sensor, an iris sensor, or an illuminance sensor) may be implemented as embedded in the display device 1460 (e.g., a display).

The processor 1420 may execute, for example, software (e.g., a program 1440) to control at least one other component (e.g., a hardware or software component) of the electronic device 1401 coupled with the processor 1420, and may perform various data processing or computation. According to one embodiment, as at least part of the data processing or computation, the processor 1420 may load a command or data received from another component (e.g., the sensor module 1476 or the communication module 1490) in volatile memory 1432, process the command or the data stored in the volatile memory 1432, and store resulting data in non-volatile memory 1434. According to an embodiment, the processor 1420 may include a main processor 1421 (e.g., a central processing unit (CPU) or an application processor (AP)), and an auxiliary processor 1423 (e.g., a graphics processing unit (GPU), an image signal processor (ISP), a sensor hub processor, or a communication processor (CP)) that is operable independently from, or in conjunction with, the main processor 1421. Additionally or alternatively, the auxiliary processor 1423 may be adapted to consume less power than the main processor 1421, or to be specific to a specified function. The auxiliary processor 1423 may be implemented as separate from, or as part of the main processor 1421.

The auxiliary processor 1423 may control at least some of functions or states related to at least one component (e.g., the display device 1460, the sensor module 1476, or the communication module 1490) among the components of the electronic device 1401, instead of the main processor 1421 while the main processor 1421 is in an inactive (e.g., sleep) state, or together with the main processor 1421 while the main processor 1421 is in an active state (e.g., executing an application). According to an embodiment, the auxiliary processor 1423 (e.g., an image signal processor or a communication processor) may be implemented as part of another component (e.g., the camera module 1480 or the communication module 1490) functionally related to the auxiliary processor 1423.

The memory 1430 may store various data used by at least one component (e.g., the processor 1420 or the sensor module 1476) of the electronic device 1401. The various data may include, for example, software (e.g., the program 1440) and input data or output data for a command related thererto. The memory 1430 may include the volatile memory 1432 or the non-volatile memory 1434.

The program 1440 may be stored in the memory 1430 as software, and may include, for example, an operating system (OS) 1442, middleware 1444, or an application 1446.

The input device 1450 may receive a command or data to be used by other component (e.g., the processor 1420) of the electronic device 1401, from the outside (e.g., a user) of the electronic device 1401. The input device 1450 may include, for example, a microphone, a mouse, a keyboard, or a digital pen (e.g., a stylus pen).

The sound output device 1455 may output sound signals to the outside of the electronic device 1401. The sound output device 1455 may include, for example, a speaker or a receiver. The speaker may be used for general purposes, such as playing multimedia or playing record, and the receiver may be used for incoming calls. According to an embodiment, the receiver may be implemented as separate from, or as part of the speaker.

The display device 1460 may visually provide information to the outside (e.g., a user) of the electronic device 1401. The display device 1460 may include, for example, a display, a hologram device, or a projector and control circuitry to control a corresponding one of the display, hologram device, and projector. According to an embodiment, the display device 1460 may include touch circuitry adapted to detect a touch, or sensor circuitry (e.g., a pressure sensor) adapted to measure the intensity of force incurred by the touch.

The audio module 1470 may convert a sound into an electrical signal and vice versa. According to an embodiment, the audio module 1470 may obtain the sound via the input device 1450, or output the sound via the sound output device 1455 or a headphone of an external electronic device (e.g., an electronic device 1402) directly (e.g., wiredly) or wirelessly coupled with the electronic device 1401.

The sensor module 1476 may detect an operational state (e.g., power or temperature) of the electronic device 1401 or an environmental state (e.g., a state of a user) external to the electronic device 1401, and then generate an electrical signal or data value corresponding to the detected state. According to an embodiment, the sensor module 1476 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 1477 may support one or more specified protocols to be used for the electronic device 1401 to be coupled with the external electronic device (e.g., the electronic device 1402) directly (e.g., wiredly) or wirelessly. According to an embodiment, the interface 1477 may include, for example, a high definition multimedia interface (HDMI), a universal serial bus (USB) interface, a secure digital (SD) card interface, or an audio interface.

A connecting terminal 1478 may include a connector via which the electronic device 1401 may be physically connected with the external electronic device (e.g., the electronic device 1402). According to an embodiment, the connecting terminal 1478 may include, for example, a HDMI connector, a USB connector, a SD card connector, or an audio connector (e.g., a headphone connector).

The haptic module 1479 may convert an electrical signal into a mechanical stimulus (e.g., a vibration or a movement) or electrical stimulus which may be recognized by a user via his tactile sensation or kinesthetic sensation. According to an embodiment, the haptic module 1479 may include, for example, a motor, a piezoelectric element, or an electric stimulator.

The camera module 1480 may capture a still image or moving images. According to an embodiment, the camera module 1480 may include one or more lenses, image sensors, image signal processors, or flashes.

The power management module 1488 may manage power supplied to the electronic device 1401. According to one embodiment, the power management module 1488 may be implemented as at least part of, for example, a power management integrated circuit (PMIC).

The battery 1489 may supply power to at least one component of the electronic device 1401. According to an embodiment, the battery 1489 may include, for example, a primary cell which is not rechargeable, a secondary cell which is rechargeable, or a fuel cell.

The communication module 1490 may support establishing a direct (e.g., wired) communication channel or a wireless communication channel between the electronic device 1401 and the external electronic device (e.g., the electronic device 1402, the electronic device 1404, or the server 1408) and performing communication via the established communication channel. The communication module 1490 may include one or more communication processors that are operable independently from the processor 1420 (e.g., the application processor (AP)) and supports a direct (e.g., wired) communication or a wireless communication. According to an embodiment, the communication module 1490 may include a wireless communication module 1492 (e.g., a cellular communication module, a short-range wireless communication module, or a global navigation satellite system (GNSS) communication module) or a wired communication module 1494 (e.g., a local area network (LAN) communication module or a power line communication (PLC) module). A corresponding one of these communication modules may communicate with the external electronic device via the first network 1498 (e.g., a short-range communication network, such as Bluetooth™ wireless-fidelity (Wi-Fi) direct, or infrared data association (IrDA)) or the second network 1499 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 1492 may identify and authenticate the electronic device 1401 in a communication network, such as the first network 1498 or the second network 1499, using subscriber information (e.g., international mobile subscriber identity (IMSI)) stored in the subscriber identification module 1496.

The antenna module 1497 may transmit or receive a signal or power to or from the outside (e.g., the external electronic device) of the electronic device 1401. According to an embodiment, the antenna module 1497 may include an antenna including a radiating element composed of a conductive material or a conductive pattern formed in or on a substrate (e.g., PCB). According to an embodiment, the antenna module 1497 may include a plurality of antennas. In such a case, at least one antenna appropriate for a communication scheme used in the communication network, such as the first network 1498 or the second network 1499, may be selected, for example, by the communication module 1490 (e.g., the wireless communication module 1492) from the plurality of antennas. The signal or the power may then be transmitted or received between the communication module 1490 and the external electronic device via the selected at least one antenna. According to an embodiment, another component (e.g., a radio frequency integrated circuit (RFIC)) other than the radiating element may be additionally formed as part of the antenna module 1497.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 1401 and the external electronic device 1404 via the server 1408 coupled with the second network 1499. Each of the electronic devices 1402 and 1404 may be a device of a same type as, or a different type, from the electronic device 1401. According to an embodiment, all or some of operations to be executed at the electronic device 1401 may be executed at one or more of the external electronic devices 1402, 1404, or 1408. For example, if the electronic device 1401 should perform a function or a service automatically, or in response to a request from a user or another device, the electronic device 1401, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 1401. The electronic device 1401 may provide the outcome, with or without further processing of the outcome, as at least part of a reply to the request. To that end, a cloud computing, distributed computing, or client-server computing technology may be used, for example.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 1440) including one or more instructions that are stored in a storage medium (e.g., internal memory 1436 or external memory 1438) that is readable by a machine (e.g., the electronic device 1401). For example, a processor (e.g., the processor 1420) of the machine (e.g., the electronic device 1401) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

An electronic device (e.g., 310 in FIG. 3, 1401 in FIG. 14) according to various embodiments may include a memory (e.g., 313 in FIG. 3, 1430 in FIG. 14), and a processor (e.g., 311 in FIG. 3, 1420 in FIG. 14) connected to the memory (1430 in FIG. 14). The memory, when executing, may store instructions that allow the processor to receive a sensing signal related to the user's breathing from a sensor (e.g., 301 in FIG. 3, 1476 in FIG. 14), obtain (or identify) a first pattern related to a change in breathing of a user over time and a second pattern related to a change in saturation of peripheral oxygen of the user over time based on the sensing signal, and may determine (or identify) an oxygen delivery time (ODT), based on a comparison between the first pattern and the second pattern.

According to various embodiments, the oxygen transfer time may be determined based on a time difference between the first pattern and the second pattern.

According to various embodiments, the sensing signal may be received when the second body part of the user contacts the sensor or is located near the sensor. The oxygen delivery time may indicate the time taken for oxygen to be delivered from the first body part of the user to the second body part of the user through the blood.

According to various embodiments, the instructions may allow the processor to further compare the first pattern and the second pattern to identify a first section of the first pattern and a second section of the second pattern that are pattern-matched, identify the time-delay value due to the difference between the start time point of the first section and the start time point of the second section, and determine the oxygen delivery time corresponding to the identified time-delay value.

According to various embodiments, the sensor may be a multiple-wavelength photoplethysmogram (PPG) sensor.

According to various embodiments, the instructions may allow the processor to further determine blood pressure information, based on the oxygen delivery time and display the same on a screen.

According to various embodiments, the first body part may correspond to the lungs, and the second body part may correspond to the finger.

According to various embodiments, the sensor may be embedded in the electronic device.

According to various embodiments, the sensor may be configured to be detachable from the electronic device.

According to various embodiments, the first pattern and the second pattern, obtained based on the sensing signal, may have the same time stamp.

According to various embodiments, the instructions may allow the processor to additionally transmit information about the first pattern and the second pattern to a server along with time-stamp information related to the sensing signal.

A method according to various embodiments may include, in a method for obtaining vital signs by an electronic device, an operation of receiving a sensing signal related to a user's breath from a sensor connected to the electronic device, an operation of obtaining (or identifying) a first pattern related to a change in breathing of the user over time and a second pattern related to a change in saturation of peripheral oxygen of the user over time, based on the sensing signal, and an operation of determining (or identifying) an oxygen delivery time (ODT), based on a comparison between the first pattern and the second pattern.

According to various embodiments, the oxygen delivery time may be determined based on a time difference between the first pattern and the second pattern.

According to various embodiments, the sensing signal may be received when the second body part of the user contacts the sensor or is located near the sensor. The oxygen delivery time may indicate the time taken for oxygen to be delivered from the first body part of the user to the second body part of the user via the blood.

According to various embodiments, the operation of determining the oxygen delivery time, based on the comparison between the first pattern and the second pattern may include an operation of comparing the first pattern and the second pattern to identify a first section of the first pattern and a second section of the second pattern that are pattern-matched, an operation of identifying a time-delay value according to the difference between the start time of the first section and the start time of the second section, and an operation of determining the oxygen delivery time corresponding to the identified time-delay value.

According to various embodiments, the sensor may be a multiple-wavelength photoplethysmogram (PPG) sensor.

According to various embodiments, an operation of determining blood pressure information, based on the oxygen delivery time and displaying the same on a screen may be further included.

According to various embodiments, the first body part may correspond to the lungs, and the second body part may correspond to the finger.

A storage medium according to various embodiments may be a non-transient computer-readable storage medium that stores instructions, wherein, when the instructions are executed by at least one processor of an electronic device, the instructions may cause the electronic device to receive a sensing signal related to the user's breathing from a sensor, obtain a first pattern related to a change in breathing of the user over time and a second pattern related to a change in saturation of peripheral oxygen of the user over time, based on the sensing signal, and determine an oxygen delivery time (ODT), based on the comparison between the first pattern and the second pattern.

According to various embodiments, the oxygen delivery time may be determined based on a time difference between the first pattern and the second pattern.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
a memory to store instructions; and
at least one processor connected to the memory,
wherein the instructions, which when executed, configure the at least one processor to:
receive a sensing signal related to respiration of a user from a sensor,
obtain a first pattern related to a change in the respiration of the user over time, and a second pattern related to a change in saturation of peripheral oxygen of the user over time based on the sensing signal, and
determine an oxygen delivery time (ODT), based on a comparison between the first pattern and the second pattern.

2. The electronic device of claim 1, wherein the ODT is determined based on a time difference between the first pattern and the second pattern.

3. The electronic device of claim 1,
wherein the sensing signal is received based on a second body part of the user contacting the sensor or being positioned near the sensor, and
wherein the oxygen delivery time indicates a time taken for oxygen to be delivered, via blood, from a first body part of the user to the second body part of the user.

4. The electronic device of claim 1, wherein the instructions, which when executed, further configure the at least one processor to:
compare the first pattern and the second pattern to identify a first section of the first pattern and a second section of the second pattern that are pattern-matched;
identify a time-delay value due to a difference between a start time point of the first section and a start time point of the second section; and
determine the oxygen delivery time corresponding to the identified time-delay value.

5. The electronic device of claim 1, wherein the sensor comprises a multiple-wavelength photoplethysmogram (PPG) sensor.

6. The electronic device of claim 1, wherein the instructions, which when executed, further configure the at least one processor to:
determine blood pressure information, based on the oxygen delivery time; and
control a display of the electronic device to display the determined blood pressure information on a screen.

7. The electronic device of claim 3,
wherein the first body part corresponds to lungs, and
wherein the second body part corresponds to a finger.

8. The electronic device of claim 1, wherein the sensor is embedded in the electronic device.

9. The electronic device of claim 1, wherein the sensor is configured to be detachable from the electronic device.

10. The electronic device of claim 1, wherein the first pattern and the second pattern, obtained based on the sensing signal, are associated with a same time stamp.

11. The electronic device of claim 1, wherein the instructions, which when executed, further configure the at least one processor to transmit information about the first pattern and the second pattern to a server along with time-stamp information related to the sensing signal.

12. A method for obtaining a vital sign by an electronic device, the method comprising:
receiving a sensing signal related to respiration of a user from a sensor connected to the electronic device;
obtaining a first pattern related to a change in respiration of the user over time, and a second pattern related to a change in saturation of peripheral oxygen of the user over time based on the sensing signal; and
determining an oxygen delivery time (ODT), based on a comparison between the first pattern and the second pattern.

13. The method of claim 12, wherein the ODT is determined based on a time difference between the first pattern and the second pattern.

14. The method of claim 12,
wherein the sensing signal is received based on a second body part of the user contacting the sensor or being positioned near the sensor, and
wherein the oxygen delivery time indicates a time taken for oxygen to be delivered, via blood, from a first body part of the user to the second body part of the user.

15. The method of claim 12, wherein the determining of the oxygen delivery time, based on the comparison between the first pattern and the second pattern comprises:
comparing the first pattern and the second pattern to identify a first section of the first pattern and a second section of the second pattern that are pattern-matched;
identifying a time-delay value according to a difference between a start time of the first section and a start time of the second section; and
determining the oxygen delivery time corresponding to the identified time-delay value.

16. The method of claim 12, wherein the sensor comprises a multiple-wavelength photoplethysmogram (PPG) sensor.

17. The method of claim 12, further comprising:
determining blood pressure information, based on the oxygen delivery time; and
displaying the determined blood pressure information on a screen.

18. The method of claim 14,
wherein the first body part corresponds to lungs, and
wherein the second body part corresponds to a finger.

19. A non-transitory computer-readable recording medium having recorded thereon at least one program comprising instructions, which when executed, configure at least one processor of an electronic device to:
receive a sensing signal related to a user's respiration from a sensor;
obtain a first pattern related to a change in respiration of the user over time, and a second pattern related to a change in saturation of peripheral oxygen of the user over time based on the sensing signal; and
determine an oxygen delivery time (ODT), based on a comparison between the first pattern and the second pattern.

20. The non-transitory computer-readable medium of claim 19, wherein the ODT is determined based on a time difference between the first pattern and the second pattern.
